(19) United States
(12) Patent Application Publication
(10) Pub. No.: US 2007/0156025 A1
(43) Pub. Date: Jul. 5, 2007

(54) Surgical retractors and methods of minimally invasive surgery

(76) Inventors: Connie Marchek, Foxboro, MA (US); William Frasier, New Bedford, MA (US); Anne Drzyzga, Taunton, MA (US); Sara Dziedzic, Braintree, MA (US); Holly Brideau, West Roxbury, MA (US); Thomas Runco, Canton, MA (US); Nicholas Pavento, Walpole, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/325,620

(22) Filed: Jan. 4, 2006

(51) Int. Cl. A61B 1/32

(52) U.S. Cl. 600/224

(57) ABSTRACT

A surgical retractor includes a plurality of blade assemblies interconnected by a plurality of racks. One or more of the blade assemblies is movable along a rack to selectively expand the retractor. At least one of the blade assemblies includes a blade that is rotatably connected to the blade assembly and that is rotatable independent of other blades of the retractor.

### BACKGROUND

In surgical procedures, it is important to minimize trauma to the patient and damage to tissue to facilitate patient recovery. One way to accomplish this is to minimize the size of the incision for the surgical procedure and minimize the cutting of tissue to access the target anatomy. A number of retractors are available that are designed to expand a small surgical incision and provide access to a surgical site. Such retractors typically include two or more retractor blades that separate to expand the incision and create an access channel through which to conduct the surgical procedure. One problem with such retractors is that the access channel of the expanded retractor is often restricted to one shape or configuration.

### SUMMARY

Disclosed herein are surgical retractors and methods of minimally invasive surgery that minimize tissue trauma and facilitate access to a surgical site. In one exemplary embodiment, a surgical retractor comprises a plurality of blade assemblies interconnected by a plurality of racks. One or more of the blade assemblies may be movable along a rack to selectively expand the retractor. At least one of the blade assemblies includes a blade that is rotatably connected to the blade assembly and that may be rotatable independent of other blades of the retractor.

In another exemplary embodiment, a surgical retractor may comprise a first blade assembly, a second blade assembly, a third blade assembly, and a fourth blade assembly. The first blade assembly may be connected by a first rack to the third blade assembly and the first blade assembly may be movable along the first rack relative to the third blade assembly. The first blade assembly may be connected by a second rack to the fourth blade assembly and the fourth blade assembly may be movable along the second rack relative to the first blade assembly. The second blade assembly may be connected by a third rack to the third blade assembly and the third blade assembly may be movable along the third rack relative to the second blade assembly. The second blade assembly may be connected by a fourth rack to the fourth blade assembly and the second blade assembly may be movable along the fourth rack relative to the fourth blade assembly. The first blade assembly may include a first blade that is rotatable independent of blades of the other blade assemblies.

In accordance with another exemplary embodiment, a surgical retractor may comprise a first blade assembly having a first blade connected thereto and a second blade assembly having a second blade connected thereto. The first blade assembly may be connected by a first rack to the second blade assembly and at least one of the first blade assembly and the second blade assembly may be movable along the first rack. The first blade assembly may be connected by a second rack to the second blade assembly and at least one of the first blade assembly and the second blade assembly may be movable along the second rack. The retractor may be adjustable between a closed configuration in which the first blade assembly and second blade assembly are proximate one another at at least the proximal end thereof and an expanded configuration in which the first blade assembly and the second blade assembly are displaced from another. The first blade may be rotatably connected to the first blade assembly and may be rotatable independent of the second blade relative to the first blade assembly. The second blade may be rotatably connected to the second blade assembly and may be rotatable independent of the first blade relative to the second blade assembly.

An exemplary method of providing minimally invasive access to spinal anatomy may comprise making an incision and inserting a plurality of blades of a retractor through the incision. The retractor may comprise a plurality of blades interconnected by a plurality of racks. The exemplary method may include advancing the distal ends of the plurality blades into proximity to the spinal anatomy with the blades in a closed configuration in which the blades a proximate each other to form a continuous approximately enclosed access channel between the skin and the distal ends of the blades. The exemplary method may further include displacing one or more of the blades from the other blades by moving the blade along a rack to expand the working channel and rotating a first one of the blades independent of the other blades about a rotation axis to expand the access channel.

### BRIEF DESCRIPTION OF THE FIGURES

These and other features and advantages of the surgical retractors and methods disclosed herein will be more fully understood by reference to the following detailed description in conjunction with the attached drawings in which like reference numerals refer to like elements through the different views. The drawings illustrate principles of the devices and methods disclosed herein and, although not to scale, show relative dimensions.

FIG. 1 is a perspective view of an exemplary embodiment of a surgical retractor, illustrating the retractor in a closed configuration;

FIG. 2 is a perspective view of the retractor of FIG. 1, illustrating the retractor in an expanded configuration;

FIG. 3 is a top view of the retractor of FIG. 1, illustrating the retractor in a closed configuration;

FIG. 4 is an exploded view of an exemplary blade assembly of the retractor of FIG. 1;

FIG. 5 is a side view in cross section of an exemplary blade assembly of the retractor of FIG. 1, illustrating the blade of the blade assembly in a first, closed position;

FIG. 6 is a side view in cross section of an exemplary blade assembly of the retractor of FIG. 1, illustrating the blade of the blade assembly in a second, expanded position;

FIG. 7 is a top view of the retractor of FIG. 1, illustrating the retractor in an expanded configuration and positioned to provide access to spinal anatomy in a posterior approach;

FIG. 8 is a top view of the retractor of FIG. 1, illustrating the retractor in an expanded configuration and positioned to provide access to spinal anatomy in a posterior approach;

FIG. 9 is a side view in cross section of an alternative embodiment of a blade assembly;

FIG. 10 is a side view in cross section of an alternative embodiment of a blade assembly;

FIG. 11 is a side view in cross section of an alternative embodiment of a blade assembly;

FIG. 12 is a side view in cross section of an alternative embodiment of a blade assembly; and

FIG. 13 is a side view in cross section of an alternative embodiment of a blade assembly.

### DETAIL DESCRIPTION OF EXEMPLARY EMBODIMENTS

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

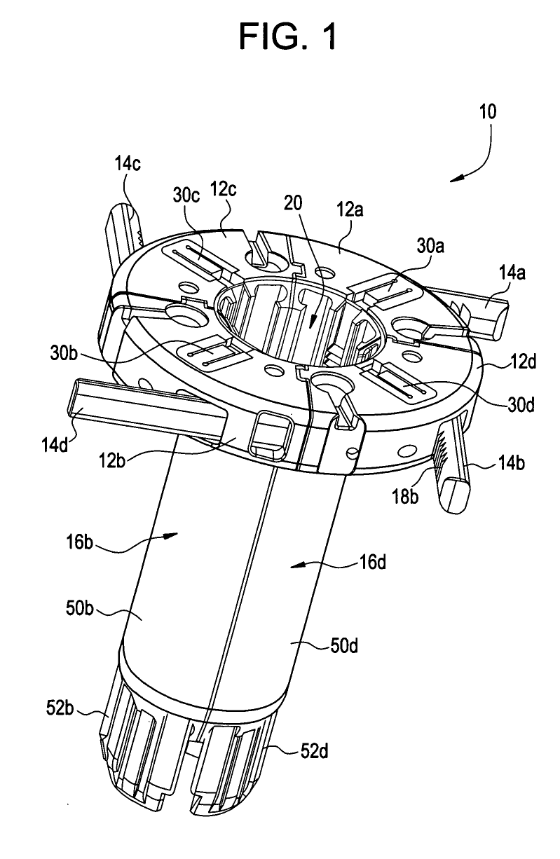

FIGS. 1-8 illustrate an exemplary embodiment of a surgical retractor 10 suitable for providing a selectively expandable access channel through which a surgical procedure may be performed on target anatomy. The exemplary surgical retractor is particularly suited for minimally invasive spine surgery and, to this end, may be inserted through a relatively small incision to provide a selectively expandable access channel from the skin to the target spinal anatomy. The exemplary surgical retractor 10 includes a plurality of blade assemblies 12 interconnected by a plurality of racks 14 allowing one or more of the blade assemblies 12 to be displaced along a rack to selectively expand the access channel. The blade assemblies 12 include tissue engaging blades 16, some or all of which may be independently rotated to allow the access channel of the retractor 10 to be selectively expanded into a variety of different shapes and sizes.

The surgical retractors disclosed herein may include a plurality of blade assemblies 12 that may include tissue engaging blades 16 that define an access channel 20 for the retractor. Any number of blade assemblies 12 may be provided. For example, a surgical retractor may include two blade assemblies, three blade assemblies, or four blade assemblies. The number (and size and shape) of blade assemblies may vary depending on, for example, the size and shape of the access channel desired, the procedure being performed, and the surgical approach, e.g. posterior, anterior, or lateral. The illustrated exemplary surgical retractor 10 includes four blade assemblies: first blade assembly 12a, second blade assembly 12b, third blade assembly 12c, and fourth blade assembly 12d.

The blade assemblies 12 of the retractors disclosed herein may be interconnected by a number of racks 14 that allow selective displacement of the blade assemblies from one another to expand the access channel of the retractor. The number of racks 14 provided can vary depending on, for example, the desired expansion of the access channel. In the illustrated embodiment, the retractor 10 includes four racks: first rack 14a, second rack 14b, third rack 14c, and fourth rack 14d. In particular, the first blade assembly 12a may be connected by the first rack 14a to the third blade assembly 12c and the first blade assembly 12a may be movable along the first rack 14a relative to the third blade assembly 12c. The first blade assembly 12a may be connected by the second rack 14b to the fourth blade assembly 12d and the fourth blade assembly 12d may be movable along the second rack 14b relative to the first blade assembly 12a. The second blade assembly 12b may be connected by the third rack 14c to the third blade assembly 12c and the third blade assembly 12c may be movable along the third rack 14c relative to the second blade assembly 12b. The second blade assembly 12b may be connected by the fourth rack 14d to the fourth blade assembly 12d and the second blade assembly 12b may be movable along the fourth rack 14d relative to the fourth blade assembly 12d.

In another exemplary embodiment, a retractor may include a first a first blade assembly having a first blade connected thereto and a second blade assembly having a second blade connected thereto. A first rack may connect the first blade assembly and the second blade assembly and the first blade assembly and/or the second blade assembly may be movable along the first rack. A second rack may connect the first blade assembly and the second blade assembly and the first blade assembly and/or the second blade assembly may be movable along the second rack.

The retractors disclosed herein may include a mechanism for selectively locking the position of a blade assembly 12 relative a rack 14. In the illustrated embodiment, for example, the retractor 10 includes a ratchet mechanism for selectively displacing a blade assembly 12 relative to a respective rack 14. Each rack 14a-d includes a plurality of teeth 18a-d extending along the length of the rack 14. Each blade assembly 12a-d includes a complementary pawl that can selectively engage the teeth 18 of a respective rack to lock the position of the blade assembly 12 relative to the rack 14, when the teeth are engaged by the pawl, or to release the blade assembly 12 from the rack 14 to permit motion along the rack 14 when the pawl is disengaged from the rack. One skilled in the art will appreciate that other mechanisms, including, for example, a screw or the like that may be selectively advanced relative to the blade assembly into contact with the respective rack, may be employed to permit selective displacement of a blade assembly relative to a rack.

The shape of a rack 14 along its longitudinal axis can be varied to provide an expanded access channel having a different size and shape. In the illustrated exemplary embodiment, for example, all of the racks 14a-d of the surgical retractor 10 are linear. In such a configuration, the blade assemblies 12a-d may be displaced along a respective rack 14a-d with each blade 16a-d remaining parallel in orientation with respect to the other blades. In alternative embodiments, one or more of the racks 14 may be arcuate along its length to permit lateral and angular expansion of the access channel or may be flexible or hinged allowing for variable angular expansion.

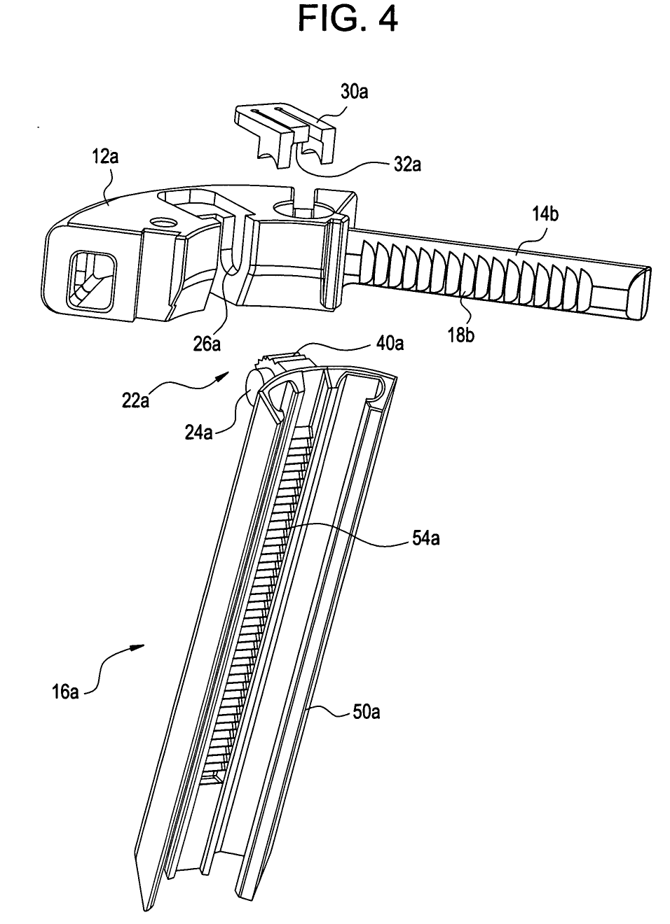

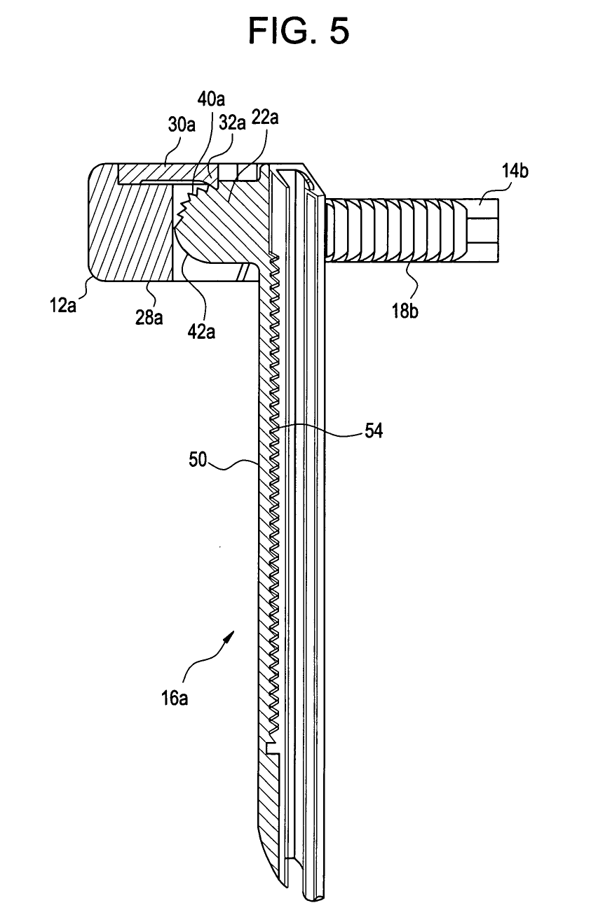

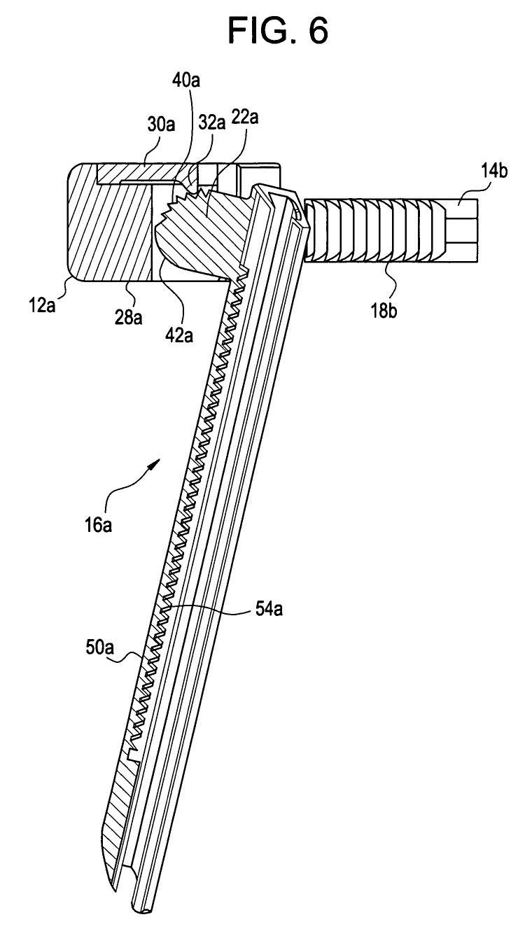

In the exemplary retractors disclosed herein, the blade 16 of one or more of the blade assemblies 12 of the retractor may be rotationally adjustable relative to the blade assembly and the blade 16 may be rotatable independent of other blades of the retractor. For example, a blade 16 may be rotationally connected to the blade assembly. In the exemplary embodiment, the blade assemblies 12a-d each include a blade 16a-d that is rotationally connected to its respective blade assembly. In such a configuration, each blade 16a-d may rotate relative to the respective blade assembly 12a-d independent of the other blades to selectively expand the access channel 20 of the retractor 10. In the illustrated embodiment, the proximal end 22 of each blade 16 can be configured to facilitate rotational connection of the blade 16 to the blade assembly 12. Referring to FIG. 4, for example, the proximal end 22a of the first blade 16a may include an integral rotation shaft 24a that may seat within a pair of grooves 26a provided in the first blade assembly 12a. The rotation shaft 24a of the first blade 16a defines a rotation axis about which the first blade 16a may rotate. In the illustrated embodiment, the rotation axis of the first blade 16a is oriented in plane that is generally parallel to the plane defined by the axis of the first rack 14a and the second rack 14b, as well as the plane defined by the bottom surface 28a of the first blade assembly 12a. The first blade 16a may rotate between a first, closed position, illustrated in FIG. 5, in which the blade 16a is oriented approximately perpendicular to a plane defined by the bottom surface 28a of the blade assembly 12a, and a second, fully expanded position in which the blade 16a is oriented at an angle other than perpendicular to plane defined by the bottom surface 28a of the blade assembly 12a. The first blade 12a may be rotated to any position between the first, closed position and the second, fully expanded position. FIG. 6 illustrates the first blade in an expanded position between the first, closed position and the second, fully expanded configuration. In the illustrated embodiment, the second, third and fourth blade assemblies 12b-d are constructed in a manner analogous to the first blade assembly 12a.

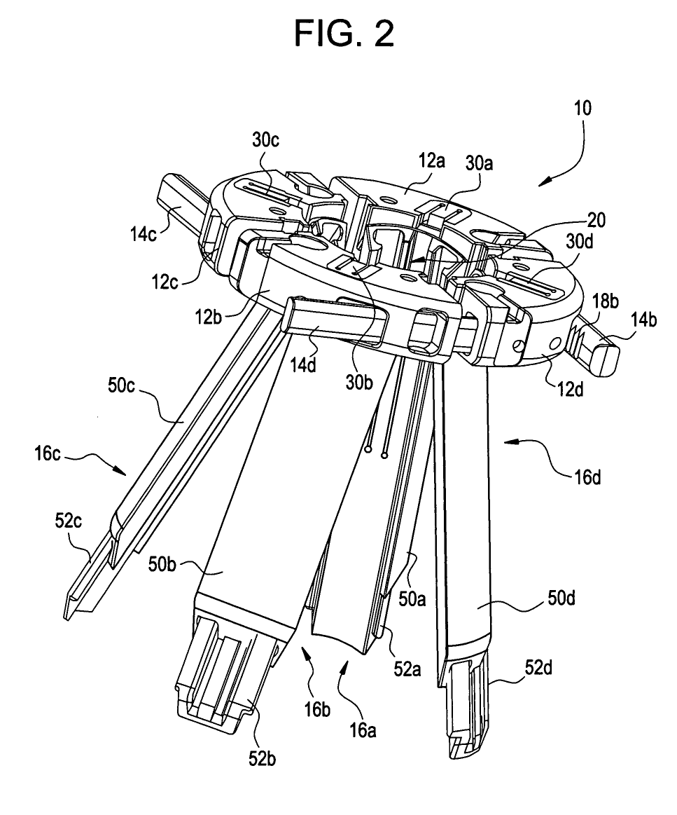

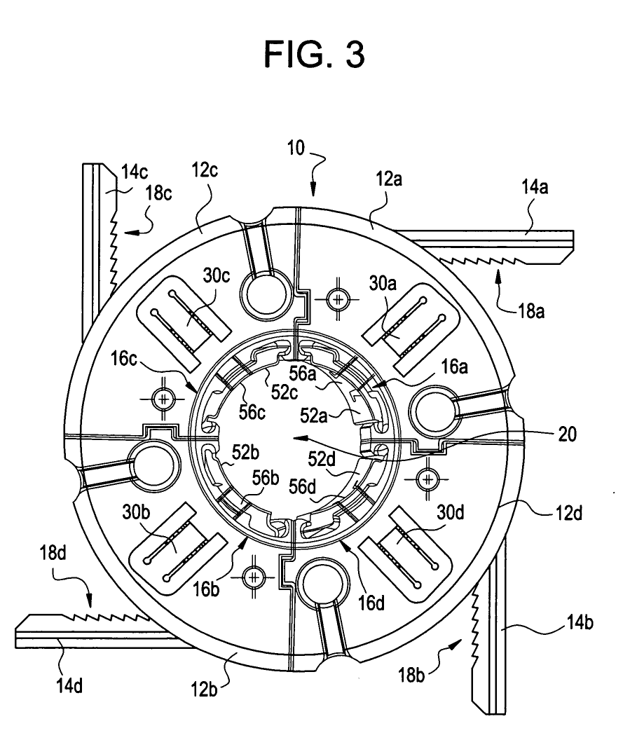

The retractors disclosed herein may be adjustable between a closed configuration, illustrated in FIGS. 1 and 3, in which the blades 16a-d of the blade assemblies 12a-d are proximate or may contact adjacent blades along at least a portion of the length of the blades 16a-d to form a continuously approximately enclosed access channel 20, and a fully expanded configuration in which the blade assemblies 12a-d are fully displaced along a respective rack 14a-d and the blades 16a-d are adjusted to the second, fully expanded position. The exemplary retractor may be expanded to any configuration between the closed configuration and the fully expanded configuration. FIG. 2 illustrates the exemplary retractor 10 in an expanded configuration between the closed configuration and the fully expanded configuration. The cross sectional size and shape of the access channel 20 in the closed configuration may vary depending on, for example, the number of blades provided, the surgical procedure being performed and the designed approach, e.g., anterior, lateral, or posterior. In the exemplary embodiment, the blades 16a-d form a cylindrical access channel 20 having a circular cross section when the blades 16a-d are in the first, closed position. The amount of rotational adjustment for the blades 16a-d between the first, closed position and the second, fully expanded position may be varied. For example, in the exemplary embodiment, each blade 16 may rotate approximately 45° between the first, closed position and the second, fully expanded position.

The retractor 10 may include a blade adjustment mechanism for selectively adjusting the rotational position of a rotationally adjustable blade. Referring to the first blade assembly 12a, for example, the blade adjustment mechanism of the exemplary retractor 10 may be a pawl 30a connected to blade assembly 12a for selectively engaging a plurality of teeth 40a provided on the proximal end 22a of a blade 16a. Each blade assembly of the exemplary retractor 10 may include an analogous adjustment mechanism, as in the illustrated retractor 10, or may have distinct blade adjustment mechanisms. Continuing to refer to first blade assembly 16a and FIGS. 4-6, for example, the pawl 30a may be a leaf spring having a tooth 32a for selectively engaging the teeth 40a on the proximal end 22a of the first blade 16a. The tooth 32a of the pawl 30a may pivot into and out of engagement with the teeth 40a provided on the proximal end 22a of the first blade 16a. The tooth 32a of the pawl 30a may be biased into engagement with the teeth 40a of the first blade 16a. The teeth 40a may be provided on an arcuate surface 42a of the proximal end 22a of the first blade 16a to facilitate rotational positioning of the first blade 16a. When the tooth 32a of the pawl 30a is engaged with the teeth 40a of the first blade 16a, the pawl 30a inhibits rotation of the first blade 16a. When the tooth 32a of the pawl 30a is pivoted out of engagement with the teeth 40a, the first blade 16a may be rotated into the desired rotational position.

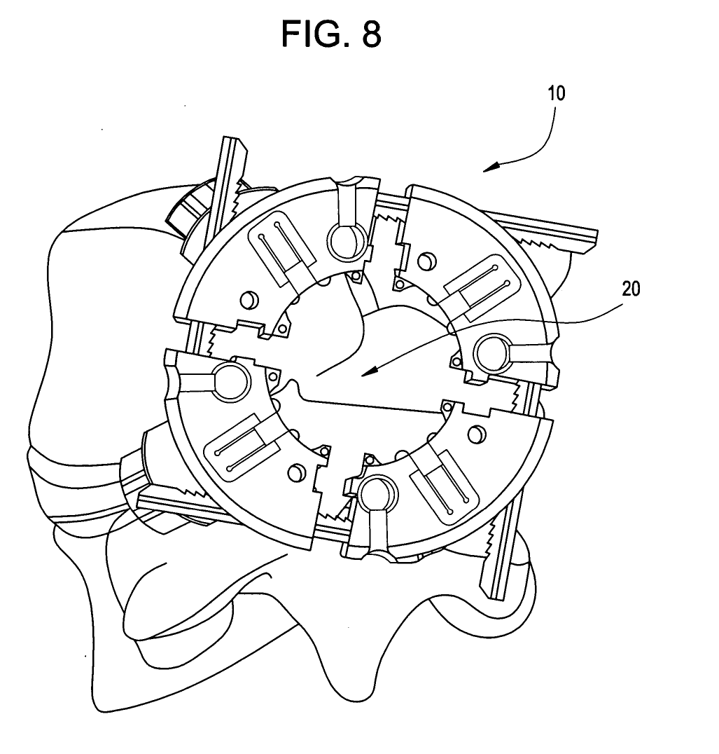

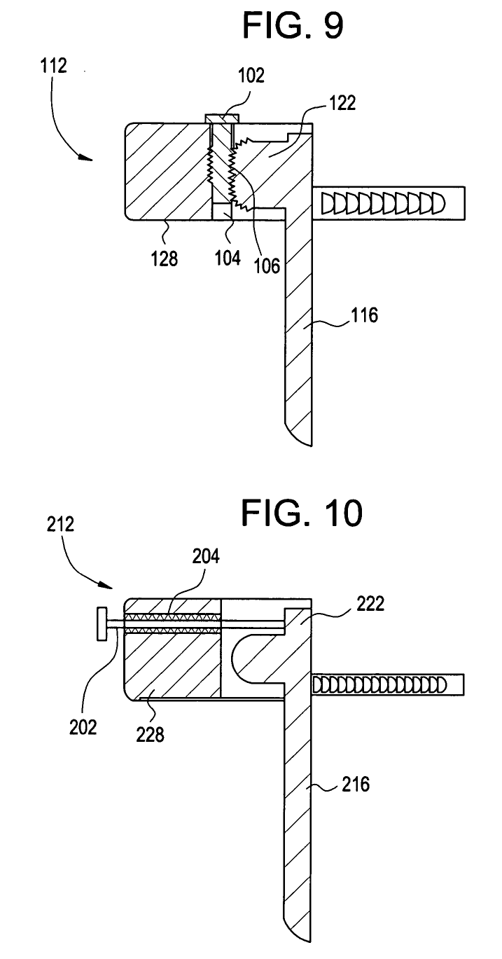

In alternative embodiments, the blade adjustment mechanism may have a different structure. Referring to FIG. 9, for example, the blade adjustment mechanism of an exemplary blade assembly 112 may include a screw 102 received within a threaded hole 104 provided in the blade assembly 112. The threads of the screw 102 engage threads 106 provided on the proximal end 122 of the blade 116. Rotation of the screw 102 relative to the blade 116 can adjust the rotational position of a rotationally adjustable blade 116. In the exemplary embodiment, the axis of the screw 102 is oriented generally perpendicular to the plane defined by the bottom surface 128 of the blade assembly 112. Rotation of the screw 102 in a first direction causes the blade 116 to rotate from a first, closed position, illustrated in FIG. 9, toward a second, fully expanded position. Rotation of the screw 102 in a second direction, opposite the first direction, causes the blade 116 to rotate from an expanded position toward the closed position.

Alternatively, the blade adjustment mechanism may include a screw received within a threaded bushing connected to the first blade. Rotation of the screw may cause the bushing to move along an axis of the screw to adjust the rotational orientation of the first blade.

Referring to FIG. 10, another exemplary embodiment of a blade adjustment mechanism is illustrated. The blade adjustment mechanism of the exemplary blade assembly 212 includes a screw 202 received within a threaded hole 204 provided in the blade assembly. The screw 202 has a screw axis that is oriented generally parallel to the plane defined by the bottom surface 228 of the blade assembly. The distal end of the screw 202 may engage the proximal end 222 of the tissue engaging blade 216. Movement of the screw 202 along a screw axis relative to the blade assembly 212 adjusts the rotational orientation of the blade 216 by rotating the blade 216 about the rotation axis of the blade 216.

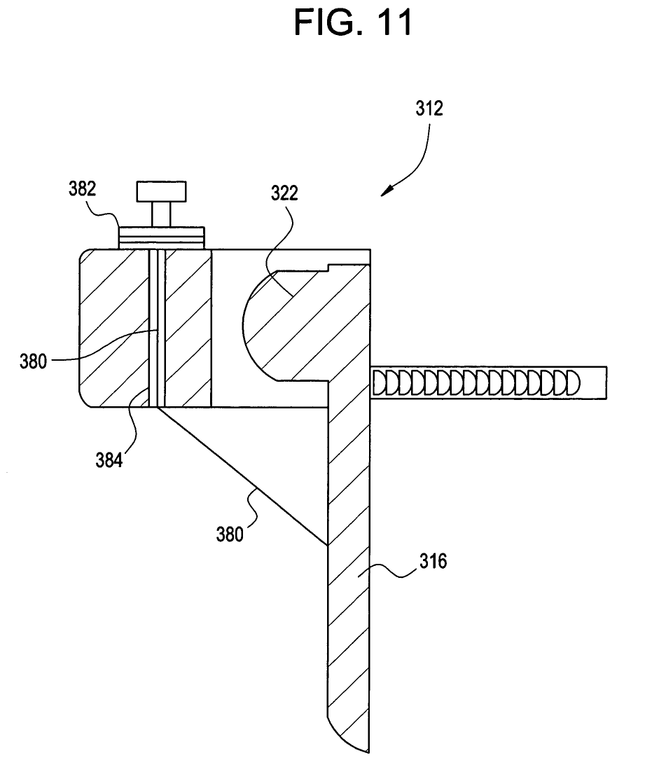

Referring to FIG. 11, another exemplary embodiment of blade adjustment mechanism is illustrated. The blade adjustment mechanism of the exemplary blade assembly 312 includes cable 380 positioned through an opening 384 in the blade assembly 312. The cable 380 may be connected at one end to a tissue engaging blade 316. At the other end, the cable 380 may be connected to a wheel 382 about which the cable 380 may be wound. Adjustment of the cable 380 along the axis of the cable 380 adjusts the rotational position of the blade 316. Rotation of the wheel 382 can cause the cable 380 to pull on the blade 316 and rotate the blade 316 about the shaft 322. A spring may be provided to bias the blade 316 to the first, closed position illustrated in FIG. 11.

Referring to FIG. 12, another exemplary embodiment of a blade adjustment mechanism is illustrated. The blade adjustment mechanism of the exemplary blade assembly 412 includes a rotatable disk 407 rotatably connected to the blade assembly 412 and engageable with the proximal end 422 of the tissue engaging blade 416. In the exemplary embodiment, the proximal end 422 of the blade 416 includes an arcuate surface for engaging the disk 407. Rotation of the disk 407 relative to the blade assembly 412 rotates the proximal end 422 of the blade 416 to adjust the rotational orientation of the blade 416. In certain exemplary embodiments, the disk 407 may be a gear having teeth for engaging teeth provided on the arcuate surface of the proximal end 422 of the blade 416.

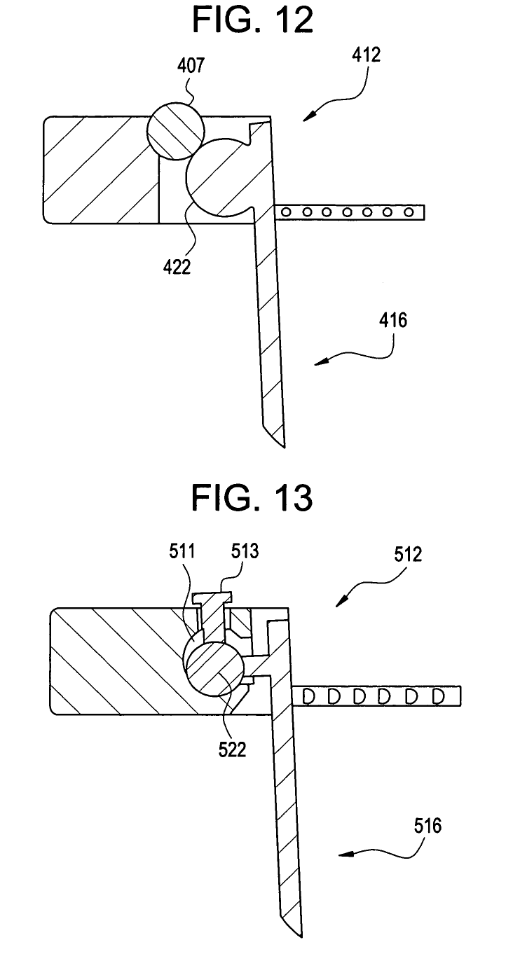

Referring to FIG. 13, another exemplary embodiment of a blade adjustment mechanism is illustrated. The blade adjustment mechanism of the exemplary blade assembly 512 includes a cavity 511 provided in the blade assembly 512 for receiving the proximal end 522 of a tissue engaging blade 516. In the exemplary embodiment, the cavity 511 has a size and shape complementary to the size and shape of the proximal end 522 of the blade 516 and selected to allow the blade 516 to rotate relative to the blade assembly 512. In the exemplary embodiment, for example, the proximal end 522 of the blade 516 may be approximately spherical in shape and the cavity 511 may include a seat that is approximately spherical in shape for engaging the proximal end 522 of the blade 516. A screw 513 or the like may be provided to fix the proximal end 522 of the blade 516 into contact with the seat of the cavity 511 and thereby inhibit rotation of the blade 516.

One skilled in the art will appreciate that other blade adjustment mechanisms may be employed to adjust the rotational position of a rotationally adjustable blade.

One or more of the blades of the retractor may have an adjustable length, e.g. the blade may telescope to selectively adjust the length of the blade. Referring to the exemplary embodiment illustration in FIGS. 1-8, for example, one or more of the blades 16 may include a primary blade 50 connected to a blade assembly 12 and an adjustable blade 52 that is operatively coupled to the primary blade 50 and is adjustable relative to the primary blade 50 along the length of the primary blade 50. In the exemplary embodiment, blades 16a-d are adjustable in length and include a respective primary blade 50a-d and a respective adjustable blade 52a-d. Exemplary tissue engaging blades having an adjustable length are disclosed in U.S. Patent Application Publication No. 2005-0137461 A1, which is incorporated herein by reference. The telescoping blades may include a mechanism for selectively adjusting the position of the adjustable blade 52 relative to the primary blade 50. For example, the primary blade 50 may include a plurality of teeth 54 extending along the longitudinal axis of the primary blade 50 and the adjustable blade 52 may include a flexible tab 56 for engaging the teeth 54 of the primary blade 50. The retractor may be inserted through an incision with the adjustable blades 52 in place, as in the case of the exemplary retractor 10 illustrated in FIGS. 1-8. Alternatively, the retractor may be inserted through an incision without the adjustable blades in place. In such embodiments, the retractor 10 may be inserted with the primary blades 50 and one or more adjustable blades may be added after insertion.

The components of the retractors disclosed herein may be manufactured from any biocompatible material including metals, such as stainless steel or titanium, polymers, or composite materials. The components, such as the blades and the frame, may be constructed from the same or different materials.

An exemplary method of providing minimally invasive access to spinal anatomy employing a retractor disclosed herein may include making a skin incision for insertion of the retractor. The incision initially may be less that the diameter of the retractor in the closed configuration (e.g., with the blades of the device in the first, closed position). The incision may be expanded to accommodate the retractor by dilation, for example, by placing one or more dilators through the incision to expand the incision in a stepwise manner. The dilators may be employed to separate or dissect the underlying tissue to the target spinal anatomy. Alternatively, the surgeon may employ his finger or the retractor to dissect the underlying tissue and to expand the initial incision.

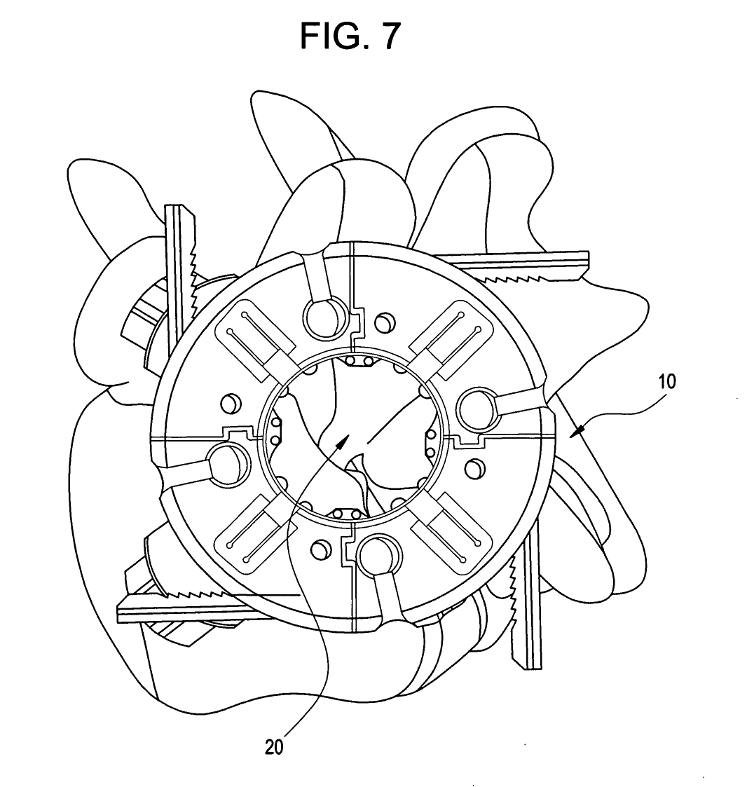

The blades of a retractor may be inserted through the incision and the distal ends of the blades may be advanced into proximity to the spinal anatomy. The blades are preferably advanced in a first, closed position, in which the blades are proximate to or contact each other to form a continuously approximately enclosed access channel between the frame, which may be located at the surface of the skin, and the distal ends of the blades. One or more of the blade assemblies may be displaced from the other blade assemblies by moving the blade assembly along a respective rack and thereby expanding the access channel. One or more of the blades may be rotated, using a blade adjustment mechanism, independent of the other blades, to selectively expand the access channel. In the case of the exemplary retractor 10, rotational adjustment of some or all of the blades of the device expands the access channel, particularly at the distal end of the access channel, thereby creating greater working space at proximate the target spinal anatomy. In addition, the length of the working channel may be increased by advancing an adjustable blade of one of the plurality of blades relative to a primary blade along a longitudinal axis of the primary blade. FIG. 7 illustrates exemplary retractor 10 in an expanded configuration in which the blades have been rotated and the retractor 10 has been positioned to provide access to spinal anatomy through a posterior approach. FIG. 8 illustrates the exemplary retractor 10 in an expanded configuration in which the blade assemblies have been displaced from one another along the racks, the blades have been rotated, and the adjustable blades have been displaced relative to the primary blades to expand the access channel.

Any number of surgical procedures may be performed through the access channel including, for example, removal of some or all of one or more discs, placement of bone fusion promoting material, placement of an spine arthroplasty device such as an artificial disc, placement of spinal implants such as hooks, rods, and screws.

After the surgical procedure is performed, the retractor may be returned to the closed configuration and removed from the incision.

While the surgical retractors and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A retractor comprising:
a first blade assembly, a second blade assembly, a third blade assembly, and a fourth blade assembly,
the first blade assembly connected by a first rack to the third blade assembly, the first blade assembly being movable along the first rack relative to the third blade assembly,
the first blade assembly connected by a second rack to the fourth blade assembly, the fourth blade assembly being movable along the second rack relative to the first blade assembly,
the second blade assembly connected by a third rack to the third blade assembly, the third blade assembly being movable along the third rack relative to the second blade assembly,
the second blade assembly connected by a fourth rack to the fourth blade assembly, the second blade assembly being movable along the fourth rack relative to the fourth blade assembly,
the first blade assembly including a first blade that is rotatable independent of blades of the other blade assemblies.

2. The retractor of claim 1, wherein at least one of the blade assemblies comprises:
a primary blade coupled to one of the racks, and
an adjustable blade operatively coupled to the primary blade and adjustable relative to the primary blade along a longitudinal axis of the primary blade.

3. The retractor of claim 2, wherein the primary blade includes a plurality of teeth extending along the longitudinal axis of the primary blade and the adjustable blade includes a flexible tab for engaging the teeth of the primary blade.

4. The retractor of claim 1, further comprising a blade adjustment mechanism for selectively adjusting the rotational position of the first blade.

5. The retractor of claim 4, wherein the blade adjustment mechanism comprises a pawl pivotally connected to the first blade assembly for selectively engaging a plurality of teeth provided at a proximal end of the first blade.

6. The retractor of claim 5, wherein the pawl is biased into contact with the teeth.

7. The retractor of claim 6, wherein the teeth are provide on an arcuate surface of the proximal end of the first blade.

8. The retractor of claim 4, wherein the blade adjustment mechanism comprises a screw received within a threaded hole provided in the first blade assembly, movement of the screw along a screw axis relative to the first blade assembly adjusts the rotational orientation of the first blade.

9. The retractor of claim 8, wherein the screw axis is oriented generally parallel to a plane defined by the bottom surface of the first blade assembly.

10. The retractor of claim 8, wherein the screw axis is oriented generally perpendicular to a plane defined by the bottom surface of the first blade assembly.

11. The retractor of claim 4, wherein the blade adjustment mechanism comprises a cable connected to the first blade and positioned through the first blade assembly, wherein adjustment of the cable along the cable axis adjusts the rotational orientation of the first blade.

12. The retractor of claim 1, wherein the second blade assembly includes a second blade that is rotatable independent of blades of the other blade assemblies.

13. The retractor of claim 12, wherein the third blade assembly includes a third blade that is rotatable independent of blades of the other blade assemblies.

14. The retractor of claim 13, wherein the fourth blade assembly includes a fourth blade that is rotatable independent of blades of the other blade assemblies.

15. The retractor of claim 1, wherein the retractor is adjustable between a closed configuration in which the blade assemblies are proximate one another at at least the proximal ends thereof and an expanded configuration in which at least one of the blade assemblies is displaced from adjacent blade assemblies.

16. The retractor of claim 15, wherein blades of the blade assemblies contact adjacent blades along a length of the blades to form a continuously enclosed working channel when the retractor is in the closed configuration.

17. The retractor of claim 16, wherein the working channel has an approximately circular cross section when the retractor is in the closed configuration.

18. The retractor of claim 15, wherein the retractor is adjustable to a fully expanded configuration in which each blade assembly is fully displaced along a respective rack.

19. A retractor comprising:
a first blade assembly having a first blade connected thereto; and
a second blade assembly having a second blade connected thereto,
a first rack connecting the first blade assembly and the second blade assembly, at least one of the first blade assembly and the second blade assembly being movable along the first rack,
a second rack connecting the first blade assembly and the second blade assembly, at least one of the first blade assembly and the second blade assembly being movable along the second rack,
the retractor being adjustable between a closed configuration in which the first blade assembly and second blade assembly are proximate one another at at least the proximal end thereof and an expanded configuration in which the first blade assembly and the second blade assembly are displaced from another,
the first blade rotatably connected to the first blade assembly and being rotatable independent of the second blade relative to the first blade assembly, and
the second blade rotatably connected to the second blade assembly and being rotatable independent of the first blade relative to the second blade assembly.

20. The retractor of claim 19, wherein first blade and the second blade contact one another along a length of the blades to form a continuously enclosed working channel when the retractor is in the closed configuration.

21. The retractor of claim 20, wherein the working channel has an approximately circular cross section when the retractor is in the closed configuration.

22. The retractor of claim 19, wherein the retractor is adjustable to a fully expanded configuration in which the first blade assembly and the second blade assembly are fully displaced from one another.

23. The retractor of claim 19, wherein the first blade and the second blade comprise
a primary blade connected to the blade assembly, and
an adjustable blade operatively coupled to the primary blade and adjustable relative to the primary blade along a longitudinal axis of the primary blade.

24. The retractor of claim 19, further comprising:
a first blade adjustment mechanism for selectively adjusting the rotational position of the first blade and
a second blade adjustment mechanism for selectively adjusting the rotational position of the second blade.

25. A method of providing minimally invasive access to spinal anatomy, the method comprising:
making an incision,
inserting a plurality of blades of a retractor through the incision, the retractor comprising a plurality of blades interconnected by a plurality of racks,
advancing the distal ends of the plurality blades into proximity to the spinal anatomy with the blades in a closed configuration in which the blades are proximate each other to form a continuous approximately enclosed access channel between the skin and the distal ends of the blades,
displacing one or more of the blades from the other blades by moving the blade along a rack to expand the working channel, and
rotating a first one of the blades independent of the other blades about a rotation axis to expand the access channel.

26. The method of claim 25, further comprising increasing the length of the working channel by advancing an adjustable blade of one of the plurality of blades relative to a primary blade along a longitudinal axis of the primary blade.

27. The method of claim 25, further comprising rotating a second one of the blades independent of the other blades about a rotation axis to expand the access channel.

28. The method of claim 25, wherein the distal ends of the blades of the retractor are advanced through a posterior approach into proximity to posterior spinal anatomy.

29. The method of claim 25, wherein the distal ends of the blades of the retractor are advanced through an anterior approach into proximity to anterior spinal anatomy.

30. The method of claim 25, further comprising performing a surgical procedure on the spinal anatomy through the access channel.

31. The method of claim 30, further comprising rotating the first blade into contact with other blades, after performing the surgical procedure, to return the retractor to the closed configuration and removing the retractor from the incision.

32. The method of claim 25, further comprising expanding the incision and inserting the plurality of blades of a retractor through the expanded incision.

33. A surgical retractor comprising:
a plurality of blade assemblies interconnected by a plurality of racks, one or more of the blade assemblies being movable along a rack to selectively expand the retractor, at least one of the blade assemblies including a blade that is rotatably connected to the blade assembly and that is rotatable independent of other blades of the retractor.

34. The retractor of claim 4, wherein the blade adjustment mechanism comprises a gear rotationally connected to the first blade assembly, wherein rotation of the gear adjusts the rotational orientation of the first blade.

35. The retractor of claim 4, wherein the blade adjustment mechanism comprises a cavity provided in the first blade assembly having an approximately spherical seat for receiving a proximal end of the first blade and a screw for selectively engaging the proximal end of the first blade and positioning the proximal end of the first blade into contact with the seat.
